(19) United States
(12) Patent Application Publication
(10) Pub. No.: US 2020/0082918 A1
(43) Pub. Date: Mar. 12, 2020

(54) SYSTEM AND METHD OF SOCIAL-BEHAVIORAL ROI CALCULATION AND OPTIMIZATION

(71) Applicant: KONINKLIJKE PHILIPS N.V., EINDHOVEN (NL)

(72) Inventors: Eran SIMHON, Boston, MA (US); Reza SHARIFI SEDEH, Malden, MA (US)

(21) Appl. No.: 16/565,533

(22) Filed: Sep. 10, 2019

Related U.S. Application Data
(60) Provisional application No. 62/730,238, filed on Sep. 12, 2018.

(51) Int. Cl. G16H 10/20; G06Q 40/06; G06F 3/0482; G06N 20/00

(52) U.S. Cl. CPC G16H 10/20; G06N 20/00; G06F 3/0482; G06Q 40/06

(57) ABSTRACT

A patient selection tool for selecting patients for a social-behavioral determinants of health (SBDoH) program, including: a graphical user interface (GUI) module configured to present a GUI to a user, receive inputs from the user including a SBDoH factor, and to select patient cohort data based upon the inputs received from the user, a machine-learning model configured to predict a key performance indicator (KPI) for each patient based upon the patient cohort data and the SBDoH factor, a success rate module configured to predict the probability of success of the SBDoH program for each patient in the patient cohort; a return on investment (ROI) module configured to determine the cost savings associated with the SBDoH program for each patient in the patient cohort based upon the cost associated with the KPI, the probability of success of the SBDoH program, and a change in the KPI associated with the SBDoH factor, and a patient selection module configured to select patients for the SBDoH program based upon the determined cost saving associated with the SBDoH program for each patient in the patient cohort.

### CROSS-REFERENCE TO PRIOR APPLICATIONS

This application claims the benefit of U.S. Provisional Application No. 62/730,238, filed on 12 Sep. 2018. This application is hereby incorporated by reference herein.

### TECHNICAL FIELD

Various exemplary embodiments disclosed herein relate generally to a system and method of social-behavioral return on investment (ROI) calculation and optimization.

### BACKGROUND

Engaging and encouraging patients to lead a healthy life style is becoming a high priority to healthcare networks in general and accountability care organizations (ACOs) in particular. Recent studies show that about 60-70% of health outcomes are determined by socio-economic, life style and environment shortly referred to as Social-Behavioral Determinants of Health (SBDoH).

In recent years, due to the shift to value-based care, healthcare networks and employers increased their investment in programs managed outside the hospital walls such as workshops for stopping smoking, weight loss, healthy nutrition, exercise and so on. In addition, they offer programs related to access to healthcare such as assistant with transportation, financial assistance for purchasing medications, and home visits/remote monitoring.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a patient selection tool for selecting patients for a social-behavioral determinants of health (SBDoH) program, including: a graphical user interface (GUI) module configured to present a GUI to a user, receive inputs from the user including a SBDoH factor and to select patient cohort data based upon the inputs received from the user, a machine-learning model configured to predict a key performance indicator (KPI) for each patient based upon the patient cohort data and the SBDoH factor, a success rate module configured to predict the probability of success of the SBDoH program for each patient in the patient cohort; a return on investment (ROI) module configured to determine the cost savings associated with the SBDoH program for each patient in the patient cohort based upon the cost associated with the KPI, the probability of success of the SBDoH program, and a change in the KPI associated with the SBDoH factor, and a patient selection module configured to select patients for the SBDoH program based upon the determined cost saving associated with the SBDoH program for each patient in the patient cohort.

Various embodiments are described, wherein the GUI further comprises a cohort pane, an actionable factors pane, and a KPI pane.

Various embodiments are described, wherein the cohort pane includes one of a chronic condition list, geographic location list, provider group list and several lists of social factors such as age, gender and marital status.

Various embodiments are described, wherein the machine learning mode determines the change in the KPI associated with the SBDoH factor by calculating the KPI for the patient with the SBDoH factor and the KPI for the patient without the SBDoH factor and calculating the difference between the KPI for the patient with the SBDoH factor and KPI for the patient without the SBDoH factor.

Various embodiments are described, wherein machine-learning module is further configured to remove patient data variables by a propensity score matching method.

Various embodiments are described, wherein the probability of success of the SBDoH program is determined based upon patient responses to survey questions.

Various embodiments are described, wherein the patient selection module is further configured to select patients for the SBDoH program based upon a set budget for the SBDoH program.

Various embodiments are described, wherein the patient selection module is further configured to select patients for the SBDoH program based upon a ROI threshold.

Further various embodiments relate to a method of selecting patients for a social-behavioral determinants of health (SBDoH) program, including: a presenting a graphical user interface (GUI) module configured to present a GUI to a user;

receiving inputs via the GUI from the user including a SBDoH factor, selecting patient cohort data based upon the inputs received from the user, predicting, by a machine-learning model, a key performance indicator (KPI) for each patient based upon the patient cohort data and the SBDoH factor, predicting, by a success rate module, the probability of success of the SBDoH program for each patient in the patient cohort; determining, by a return on investment (ROI) module, the cost savings associated with the SBDoH program for each patient in the patient cohort based upon the cost associated with the KPI, the probability of success of the SBDoH program, and a change in the KPI associated with the SBDoH factor and selecting, by a patient selection module, patients for the SBDoH program based upon the determined cost saving associated with the SBDoH program for each patient in the patient cohort.

Various embodiments are described, wherein the GUI further comprises a cohort pane, an actionable factors pane, and a KPI pane.

Various embodiments are described, wherein the cohort pane includes one of a chronic condition list, and social factors such as age, gender, provider group, geographic location, and marital status.

Various embodiments are described, wherein determining the change in the KPI associated with the SBDoH factor further includes calculating the KPI for the patient with the SBDoH factor and the KPI for the patient without the SBDoH factor and calculating the difference between the KPI for the patient with the SBDoH factor and KPI for the patient without the SBDoH factor.

Various embodiments are described, further includes removing patient data variables by a propensity score matching method before predicting the KPI.

Various embodiments are described, wherein the probability of success of the SBDoH program is determined based upon patient responses to survey questions.

Various embodiments are described, wherein selecting patients for the SBDoH program is based upon a set budget for the SBDoH program.

Various embodiments are described, wherein selecting patients for the SBDoH program is based upon a ROI threshold.

Further various embodiments relate to a non-transitory machine-readable storage medium encoded with instructions for selecting patients for a social-behavioral determinants of health (SBDoH) program, the non-transitory machine-readable storage medium including: instructions for a presenting a graphical user interface (GUI) module configured to present a GUI to a user, instructions for receiving inputs via the GUI from the user including a SBDoH factor, instructions for selecting patient cohort data based upon the inputs received from the user, instructions for predicting, by a machine-learning model, a key performance indicator (KPI) for each patient based upon the patient cohort data and the SBDoH factor, instructions for predicting, by a success rate module, the probability of success of the SBDoH program for each patient in the patient cohort; instructions for determining, by a return on investment (ROI) module, the cost savings associated with the SBDoH program for each patient in the patient cohort based upon the cost associated with the KPI, the probability of success of the SBDoH program, and a change in the KPI associated with the SBDoH factor, and instructions for selecting, by a patient selection module, patients for the SBDoH program based upon the determined cost saving associated with the SBDoH program for each patient in the patient cohort.

Various embodiments are described, wherein the GUI further includes a cohort pane, an actionable factors pane, and a KPI pane.

Various embodiments are described, wherein the cohort pane includes one of a chronic condition list, and social factors such as age, gender, provider group, geographic location, and marital status.

Various embodiments are described, wherein instructions for determining the change in the KPI associated with the SBDoH factor further comprises instructions for calculating the KPI for the patient with the SBDoH factor and the KPI for the patient without the SBDoH factor and instructions for calculating the difference between the KPI for the patient with the SBDoH factor and KPI for the patient without the SBDoH factor.

Various embodiments are described, further includes instructions for removing patient data variables by a propensity score matching method before predicting the KPI.

Various embodiments are described, wherein the probability of success of the SBDoH program is determined based upon patient responses to survey questions.

Various embodiments are described, wherein selecting patients for the SBDoH program is based upon a set budget for the SBDoH program.

Various embodiments are described, wherein selecting patients for the SBDoH program is based upon a ROI threshold.

### BRIEF DESCRIPTION OF THE DRAWINGS

In order to better understand various exemplary embodiments, reference is made to the accompanying drawings, wherein:

FIG. 1 illustrates a flow diagram of the patient selection tool;

FIG. 2 illustrates an example of a GUI implemented by the GUI module; and

FIG. 3 is a histogram of the difference between the probabilities of having ED visit next year of smokers if they stop smoking and if they do not stop smoking.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

The shift toward value-based care forces healthcare networks to reduce costs by improving healthcare key performance indicators (KPIs) such as avoidable emergency department (ED) visits and re-admissions to the hospital. One way to reduce costs and improve healthcare KPIs is by encouraging and engaging patients to lead a healthy life style. Such an initiative has two main challenges: 1) selecting the patients that will benefit from such interventions the most; and 2) computing return on investment (ROI) of such initiatives.

The embodiments described herein disclose a patient selection tool that allows the user (e.g., a director of care management who may be a health administrator or health professional) to choose a specific cohort of patients, a social-behavioral program, and a KPI to be improved. Then, the patient selection tool will identify which patients will benefit the most from the selected program. Then, for a given budget, the patient selection tool will predict the ROI of the program. Machine learning and optimization techniques are used to predict the outcomes of patients with and without applying the selected SBDoH program. By doing so, it can predict which patients are likely to have bigger healthcare outcome improvement.

While the impact of a healthy life style on healthcare outcomes has been extensively studied at the population level, there is lack of knowledge of the impact at the specific patient level. Given the medical history of a patient and his/her social status and demographics, it is unknown how changing a specific behavior such as quitting smoking, eating healthier food, reducing alcohol consumption, etc. will impact the health outcome of this specific patient. Furthermore, the ROI of such investments is usually unpredictable and, most of the time, is unknown even retrospectively. Thus, budget allocations and decisions on who should be referred to each program is currently done manually and sometimes arbitrary.

In the embodiments described herein, a patient selection tool is described that automatically identifies which patients will most benefit from a specific social/behavioral intervention and translate it to ROI. This may then automatically be translated to referrals or may be used as a decision support tool for care managers when referring patients to SBDoH programs.

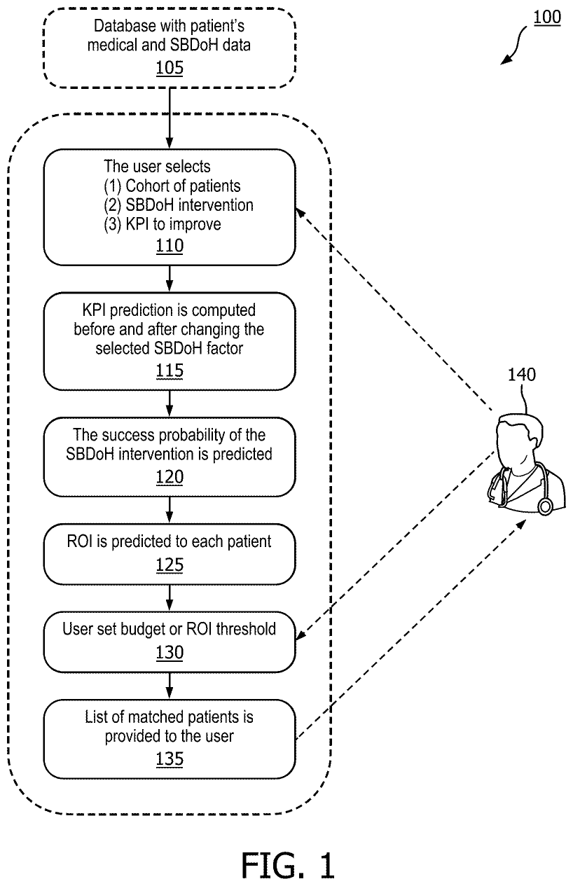

The patient selection tool includes a graphical user interface (GUI) module, a machine-learning module, a success rate module, an ROI module, and a patient selection module. FIG. 1 illustrates a flow diagram of the patient selection tool. Each of the elements of the patient selection tool will be described in further detail below.

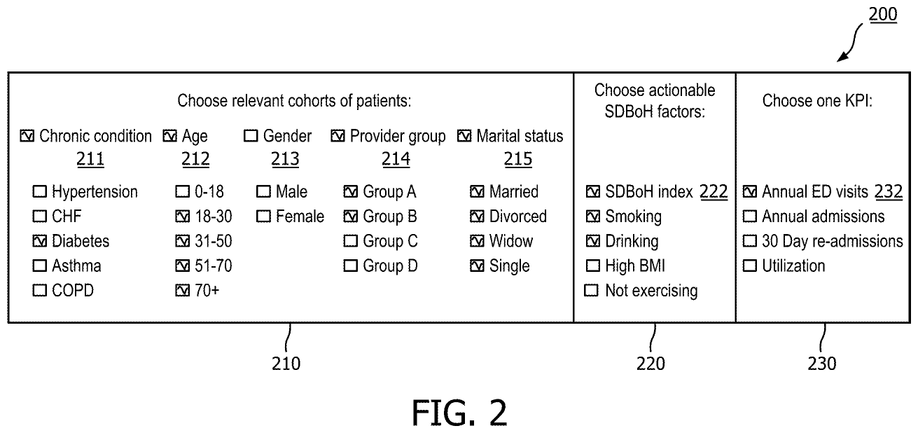

FIG. 2 illustrates an example of a GUI 200 implemented by the GUI module. In the GUI module, the user 140 chooses: (1) a specific cohort of patients based on medical condition and demographic factors; (2) a specific SBDoH factor(s) which the user wants to address; (3) a specific KPI which the user wishes to improve 110. The GUI 200 may include a cohort pane 210, an actionable factors pane 220, and a KPI pane 230. The cohort pane 210 allows the user 140 to select a cohort of patients for inclusion in a SBDoH plan. Various criteria such as chronic condition 211, age 212, gender 213, provider group 214, or marital status 215 may be used. For example, a specific chronic condition may be identified such as hypertension, congestive heart failure (CHF), diabetes, asthma, or chronic obstructive pulmonary disease (COPD), but other chronic conditions may be included as well. The GUI 200 may be configured to allow the selection of only one condition or multiple conditions. Also, if no specific condition is selected, then the chronic condition is not a factor in selecting the patient cohort. For the age criteria 212, various age ranges may be present, and again one or more age ranges or no age ranges may be selected. The gender criteria 213 allows the users to limit the patient cohort based upon gender. The provider group criteria 214 may indicate certain medical providers, medical facilities, or other grouping of patients based upon medical providers. For example, the provider groups could be based upon medical specialties. Finally, the marital status criteria 215 includes various marital statuses that may be used to determine the patient cohort. While a specific example of selection criteria have been given, other various criteria including any factor included in the EMR data may be used as well according the goals of the SBDoH program. Further, it is noted that the various criteria may be inter-related in that, if for example a specific chronic condition is selected, then only certain provider groups may be selected.

The actionable factors pane 220 may include a list of actionable SBDoH factors 222 to be considered such as, for example, SBDoH index, smoking, drinking, high body mass index (BMI), and not exercising. The user 140 may select one or multiple of these actionable SBDoH factors for use in selecting the patient cohort. Other actionable SBDoH factors may be included as well. Also, the specific SBDoH factors displayed for selection may depend on other patient selections such as when certain chronic conditions are selected.

The KPI pane 230 may include a list of KPIs 232 to be considered such as, for example, annual ED visits, annual admissions, 30-day re-admissions, and utilization. The user 140 may select one or multiple of these KPIs for use in selecting and evaluating the patient cohort. Other actionable KPIs may be included as well. Further, the specific KPIs displayed for selection may depend on other patient selections such as when certain chronic conditions are selected.

Once the user selects the various criteria in the GUI 200, the patient selection tool 100 extracts 110 a patient cohort and their associated medical data from a patient database 105. This data will then be further used by the patient selection tool 100.

The machine-learning module receives the patient data for patient cohort selected by the user 140 and first predicts the selected KPI for each patient in the selected cohort using regression/logistic regression model, based on all current data for that patient 115. Then, the patient selection tool 100 repeats calculating the selected KPI for each patient, but changing the selected SBDoH factor 115. For example, if the selected factor is smoking, the patient selection tool 100 first computes the predicted number of ED visits of all patients belonging to the selected cohort. Then, for all smokers, the patient selection tool 100 changes the smoking status to not smoking and predicts the KPI for each patient again. Finally, the patient selection tool 100 subtracts the second prediction from the first prediction. Hence, now it may be predicted how much changing the selected SBDoH factor will affect the selected KPI.

As an example, the prediction model for the KPI of the probability of an ED visit in the next year may be as follows:

$$Prob ( ED visit ) = \frac{1}{1 + {e}^{( z )}}$$

$$z = 0.2 * diabetes + 0.1 * CHF + 0.3 * smoking + 0.2 * obese …$$

where each variable (diabetes, CHF, etc.) is equal to one if the patient has this condition and to zero otherwise.

A challenge with this approach is that some of the predicting variables might be highly correlated with the selected SBDoH factor. To overcome this issue, a propensity score matching algorithm may be used to remove variables that can predict who has the selected SBDoH factor and who does not. This may be done in the following way: the identified response variable is removed from the data set and logistic regression is used to predict the SBDoH factor (for example, it is attempted to predict if a patient is smoking or not). If the area under the curve (AUC) prediction is high, it means that patients not only differ by their smoking status but by other factors as well. In this case, some of the patients and/or variables will be removed until an AUC close to 0.5 to obtained.

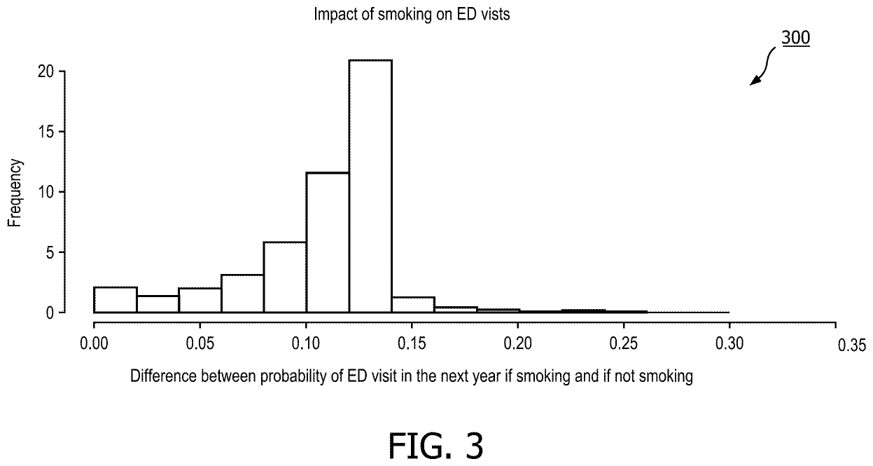

FIG. 3 is a histogram of the difference between the probabilities of having ED visit next year of smokers if they stop smoking and if they do not stop smoking. As can be seen in the histogram 300, for some patients, stopping smoking is not expected to affect their chance of having ED visits while for other patients this probability can be reduced by 30%. The vertical axis of the histogram plot shows the number of patients associated with each range of probabilities shown along the horizontal axis. As can be seen, there is a small number of patients for whom quitting smoking will reduce the probability of an ED visit in the next year by 15% to 30%. Accordingly, these patients would be the most likely to benefit from a SBDoH program.

The success rate module predicts the success probability of the selected SBDoH intervention 120, for example, the probability that a patient will stop smoking upon participation in a smoking cessation workshop. This may be done using a short questionnaire such as the following:

| | | Disagree | | | Agree |
|---|---|---|---|---|---|
| Question | N/A | Strongly | Disagree | Agree | Strongly |
| Taking an active role in my own heal care is the | 0 | −5 | 0 | 4 | 6 |
| most important thing that affects my health | | | | | |
| I am confident that I will successfully quit smoking | 0 | −7 | −2 | 0 | 2 |
| I understand my health problems and what causes | −4 | −3 | −2 | 0 | 2 |
| them. | | | | | |
| I am confident that I can maintain lifestyle | −5 | 4 | 4 | 4 | 8 |
| changes, like eating right and exercising, even | | | | | |
| during times of stress | | | | | |

| | Score | | | | | | | | |
|---|---|---|---|---|---|---|---|---|---|
| | ≤6 | 7 | 8 | 9 | 10 | 11 | 12 | 13 | 14 |
| Probability | ≤1.8% | 4.7% | 11.9.% | 26.9% | 50.0% | 73.1% | 88.1% | 95.3% | ≥98.2 |
| of Success | | | | | | | | | |

A mixed integer programing optimization technique may be used to find which questions have the best prediction power and what should be the score for each answer. Associated with each question in the table above is a score value for each answer to each question. The values associated with a patient's answers are summed to determine the patient's score. The second table shows the probability of success for different score values.

The ROI module predicts the cost of an event of the selected KPI 125, for example, the cost of an ED visit and multiplies this cost by the results from the machine-learning module and the success rate module. This results in the expected cost that will be saved if the patient will be assigned to the selected SBDoH intervention. For example, assume that the selected KPI is ED visits; the selected SBDoH intervention is a stop smoking workshop; the ED cost is $1500 on average. Also assume that for a given smoker patient, the prediction is that they will have 1.5 ED visits if they keep smoking and 0.8 ED visits if they stop smoking and assume that the success probability of the stop smoking workshop is 0.6. The expected ROI, for this patient, is 1500*(1.5-0.8)*0.6=$630. These values may then be calculated for each of the patients in the patient cohort.

Finally, the patient selection module finds the set of patients that will benefit the most from the selected SBDoH intervention based upon the output from ROI module. There are various ways that the patients may be selected for the SBDoH intervention. In one setting, the user 140 can set a budget and the system will provide a list of patients that will benefit the most from it where the size of the list is determined by the budget 135. In another setting, the user can set a ROI threshold and the system will provide a list of patients with predicted ROI higher than the threshold 135. Once the list of patients is determined, this list of patients may be presented to the user 140 using the GUI or sent to the user using other electronic means.

The embodiments described herein solve the technological problem of selecting patients for an SBDoH intervention such that the success may be predicted and the costs associated with the intervention and the ROI of the intervention are determined. These embodiments allow for a care giver to determine how to best utilize funds for SBDoH programs in order to obtain the most success from such programs.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, cloud computing systems, or other similar devices.

The memory may include various memories such as, for example L1, L2, or L3 cache or system memory. As such, the memory may include static random-access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above including implementing the GUI module, the machine-learning module, the success rate module, the ROI module, and the patient selection module.

Further such embodiments may be implemented on multiprocessor computer systems, distributed computer systems, and cloud computing systems. For example, the embodiments may be implemented as software on a server, a specific computer, on a cloud computing, or other computing platform.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A patient selection tool for selecting patients for a social-behavioral determinants of health (SBDoH) program, comprising:
a graphical user interface (GUI) module configured to present a GUI to a user, receive inputs from the user including a SBDoH factor and to select patient cohort data based upon the inputs received from the user,
a machine-learning model configured to predict a key performance indicator (KPI) for each patient based upon the patient cohort data and the SBDoH factor,
a success rate module configured to predict the probability of success of the SBDoH program for each patient in the patient cohort;
a return on investment (ROI) module configured to determine the cost savings associated with the SBDoH program for each patient in the patient cohort based upon the cost associated with the KPI, the probability of success of the SBDoH program, and a change in the KPI associated with the SBDoH factor, and
a patient selection module configured to select patients for the SBDoH program based upon the determined cost saving associated with the SBDoH program for each patient in the patient cohort.

2. The patient selection tool of claim 1, wherein the GUI further comprises a cohort pane, an actionable factors pane, and a KPI pane.

3. The patient selection tool of claim 1, wherein the cohort pane includes one of a chronic condition list, geographic location list, provider group list and several lists of social factors such as age, gender and marital status.

4. The patient selection tool of claim 1, wherein the machine learning mode determines the change in the KPI associated with the SBDoH factor by calculating the KPI for the patient with the SBDoH factor and the KPI for the patient without the SBDoH factor and calculating the difference between the KPI for the patient with the SBDoH factor and KPI for the patient without the SBDoH factor.

5. The patient selection tool of claim 1, wherein machine-learning module is further configured to remove patient data variables by a propensity score matching method.

6. The patient selection tool of claim 1, wherein the probability of success of the SBDoH program is determined based upon patient responses to survey questions.

7. The patient selection tool of claim 1, wherein the patient selection module is further configured to select patients for the SBDoH program based upon a set budget for the SBDoH program.

8. The patient selection tool of claim 1, wherein the patient selection module is further configured to select patients for the SBDoH program based upon a ROI threshold.

9. A method of selecting patients for a social-behavioral determinants of health (SBDoH) program, comprising:
a presenting a graphical user interface (GUI) module configured to present a GUI to a user,
receiving inputs via the GUI from the user including a SBDoH factor,
selecting patient cohort data based upon the inputs received from the user,
predicting, by a machine-learning model, a key performance indicator (KPI) for each patient based upon the patient cohort data and the SBDoH factor,
predicting, by a success rate module, the probability of success of the SBDoH program for each patient in the patient cohort;
determining, by a return on investment (ROI) module, the cost savings associated with the SBDoH program for each patient in the patient cohort based upon the cost associated with the KPI, the probability of success of the SBDoH program, and a change in the KPI associated with the SBDoH factor, and
selecting, by a patient selection module, patients for the SBDoH program based upon the determined cost saving associated with the SBDoH program for each patient in the patient cohort.

10. The method of claim 9, wherein the GUI further comprises a cohort pane, an actionable factors pane, and a KPI pane.

11. The method of claim 9, wherein the cohort pane includes one of a chronic condition list, and social factors such as age, gender, provider group, geographic location, and marital status.

12. The method of claim 9, wherein determining the change in the KPI associated with the SBDoH factor further comprises calculating the KPI for the patient with the SBDoH factor and the KPI for the patient without the SBDoH factor and calculating the difference between the KPI for the patient with the SBDoH factor and KPI for the patient without the SBDoH factor.

13. The method of claim 9, further comprises removing patient data variables by a propensity score matching method before predicting the KPI.

14. The method of claim 9, wherein the probability of success of the SBDoH program is determined based upon patient responses to survey questions.

15. The method of claim 9, wherein selecting patients for the SBDoH program is based upon a set budget for the SBDoH program.

16. The method of claim 9, wherein selecting patients for the SBDoH program is based upon a ROI threshold.

17. A non-transitory machine-readable storage medium encoded with instructions for selecting patients for a social-behavioral determinants of health (SBDoH) program, the non-transitory machine-readable storage medium comprising:
instructions for a presenting a graphical user interface (GUI) module configured to present a GUI to a user,
instructions for receiving inputs via the GUI from the user including a SBDoH factor,
instructions for selecting patient cohort data based upon the inputs received from the user,
instructions for predicting, by a machine-learning model, a key performance indicator (KPI) for each patient based upon the patient cohort data and the SBDoH factor,
instructions for predicting, by a success rate module, the probability of success of the SBDoH program for each patient in the patient cohort;
instructions for determining, by a return on investment (ROI) module, the cost savings associated with the SBDoH program for each patient in the patient cohort based upon the cost associated with the KPI, the probability of success of the SBDoH program, and a change in the KPI associated with the SBDoH factor; and
instructions for selecting, by a patient selection module, patients for the SBDoH program based upon the determined cost saving associated with the SBDoH program for each patient in the patient cohort.

18. The non-transitory machine-readable storage medium of claim 17, wherein the GUI further comprises a cohort pane, an actionable factors pane, and a KPI pane.

19. The non-transitory machine-readable storage medium of claim 17, wherein the cohort pane includes one of a chronic condition list, and social factors such as age, gender, provider group, geographic location, and marital status.

20. The non-transitory machine-readable storage medium of claim 17, wherein instructions for determining the change in the KPI associated with the SBDoH factor further comprises instructions for calculating the KPI for the patient with the SBDoH factor and the KPI for the patient without the SBDoH factor and instructions for calculating the difference between the KPI for the patient with the SBDoH factor and KPI for the patient without the SBDoH factor.

21. The non-transitory machine-readable storage medium of claim 17, further comprises instructions for removing patient data variables by a propensity score matching method before predicting the KPI.

22. The non-transitory machine-readable storage medium of claim 17, wherein the probability of success of the SBDoH program is determined based upon patient responses to survey questions.

23. The non-transitory machine-readable storage medium of claim 17, wherein selecting patients for the SBDoH program is based upon a set budget for the SBDoH program.

24. The non-transitory machine-readable storage medium of claim 17, wherein selecting patients for the SBDoH program is based upon a ROI threshold.
